(19) United States
(12) Patent Application Publication
(10) Pub. No.: US 2023/0136078 A1
(43) Pub. Date: May 4, 2023

(54) ANIMAL-DERIVED BACTERIA ACCELERATED CULTURE DEVICE WITH A DETECTION DEVICE

(71) Applicant: ANIMAL PRODUCTS QUALITY AND SATETY CENTER OF SHANDONG PROVINCE, JINAN (CN)

(72) Inventors: SHAONING LIU, JINAN (CN); TENG GUO, JINAN (CN); ZHI CHEN, JINAN (CN); BING SHAO, JINAN (CN); YANLIN BU, JINAN (CN); LIANGZHI ZHU, JINAN (CN); QI ZHANG, JINAN (CN); CHENGJUN ZHANG, JINAN (CN); XIUGUANG FENG, JINAN (CN); WEI ZHANG, JINAN (CN); QING LI, JINAN (CN); XIAOFEI LIU, JINAN (CN); YUNFENG HOU, JINAN (CN)

(21) Appl. No.: 17/898,063

(22) Filed: Aug. 29, 2022

(30) Foreign Application Priority Data
Nov. 3, 2021 (CN) 2021112959989

(51) Int. Cl. C12M 1/34; C12M 1/12

(52) U.S. Cl. CPC C12M 41/12; C12M 23/06; C12M 41/26; C12M 41/36

(57) ABSTRACT

An animal-derived bacteria accelerated culture device includes: a bacteria accelerated incubator; support fixed feet installed on a lower end of the bacteria accelerated incubator; limit fixed frames installed on an end of the bacteria accelerated incubator facing away from the support fixed feet, each the limit fixed frame being provided with a limit fixed groove and a first fixed pin hole; a detection frame installed on an upper end of the bacteria accelerated incubator. By controlling working detection heads to detect bacteria inside the bacteria accelerated incubator, detection results are transmitted to a processing work case through detection positioning brackets for analysis and processing, and data is displayed by a data display device, the staff can adjust parameters such as temperature, potential of hydrogen (pH), oxygen, nutrients, etc. of the bacteria accelerated incubator through displayed values, thus improving the work quality and efficiency.

### TECHNICAL FIELD

The disclosure relates to a bacteria field, in particular to an animal-derived bacteria accelerated culture device with a detection device.

### DESCRIPTION OF RELATED ART

Bacteria is one of main groups of organisms, belonging to a bacterial domain. It is also the most numerous of all living things with an estimated 5*10³⁰ in total. A shape of bacteria is quite diverse, mainly spherical, rod-shaped and spiral.

At present, an existing animal-derived bacteria accelerated culture device is simple in structure and generally does not have its own detection device, which leads to poor detection of the quality of animal-derived bacteria, thus reducing the work quality and use performance. Therefore, those skilled in the art provides an animal-derived bacteria accelerated culture device with a detection device.

### SUMMARY OF THE DISCLOSURE

A purpose of the disclosure is to provide an animal-derived bacteria accelerated culture device with a detection device, so as to solve the problems raised in the above-mentioned description of related art.

In order to solve the above problems, the disclosure provides the following technical solutions.

An animal-derived bacteria accelerated culture device with a detection device includes: a bacteria accelerated incubator, a lower end of the bacteria accelerated incubator is installed with four support fixed feet, an end of the bacteria accelerated incubator facing away from the four support fixed feet is installed with limit fixed frames, an upper end of each the limit fixed frame is provided with a limit fixed groove, and an end of each the limit fixed frame is provided with a first fixed pin hole, an upper end of the bacteria accelerated incubator is installed with a detection frame, and an end of the detection frame facing away from the bacteria accelerated incubator is installed with a processing work case.

In an embodiment, a guide fixed pin is installed inside the first fixed pin hole, each the guide fixed pin is matched with the corresponding first fixed pin hole, so as to make the detection frame be stably installed on the upper end of the bacteria accelerated incubator, thus improving the stability performance.

In an embodiment, a front end of the processing work case is installed with an adjustment control panel, a front end of the adjustment control panel is installed with a data display device, and a lower end of the data display device is installed with adjustment control buttons, so as to make an interior of the bacteria accelerated incubator have a good bacteria detection function, thus improving the use performance and work quality.

In an embodiment, an end of the detection frame facing away from the processing work case is installed with four limit fixed blocks, each the limit fixed block is matched with the corresponding limit fixed groove, so as to make the detection frame be stably installed at the upper end of the bacteria accelerated incubator, thus improving the stability performance.

In an embodiment, an end of each the limit fixed block is provided with a second fixed pin hole, and each the second fixed pin hole is matched with the corresponding guide fixed pin, so as to make the detection frame be stably installed at the upper end of the bacteria accelerated incubator, thus improving the stability performance.

In an embodiment, a lower end of the detection frame is installed with four detection positioning brackets, each the detection positioning bracket is matched with a depth inside the bacteria accelerated incubator (i.e., a length of the detection positioning bracket is no greater than the depth inside the bacteria accelerated incubator), so as to make the interior of the bacteria accelerated incubator have a good bacteria detection function, thus improving the use performance and work quality.

In an embodiment, an end of each the detection positioning bracket facing away from the detection frame is installed with a working detection head, so as to make the interior of the bacteria accelerated incubator have a good bacteria detection function, thus improving the use performance and work quality.

Compared with the related art, beneficial effects of the disclosure are as follows.

1. In the disclosure, each the limit fixed block is inserted into the corresponding limit fixed groove at the end of the limit fixed frame until each the second fixed pin hole is aligned with the corresponding first fixed pin hole, and then the corresponding guide fixed pin is inserted through the second fixed pin hole and the first fixed pin hole, so as to facilitate to lift the device by fixing adjustment positions, thus improving the use performance, and the working detection heads at the lower end of the detection positioning brackets enter the interior of the bacteria accelerated incubator, the rapid installation of the detection frame and the bacteria accelerated incubator can be completed, thus improving the work efficiency.

2. In the disclosure, by controlling the working detection heads to detect bacteria inside the bacteria accelerated incubator, detection results are transmitted to the processing work case through the detection positioning brackets for analysis and processing, and then data is displayed by the data display device, thus the staff can adjust (e.g., increase) parameters such as corresponding temperature, potential of hydrogen (pH), oxygen, nutrients, etc. of the bacteria accelerated incubator through displayed values, thereby improving the work quality and efficiency.

### BRIEF DESCRIPTION OF THE DRAWINGS

FIG. 1 illustrates a schematic structural diagram of an animal-derived bacteria accelerated culture device provided by the disclosure.

FIG. 2 illustrates a schematic structural diagram of a bacteria accelerated incubator provided by the disclosure.

FIG. 3 illustrates a schematic structural diagram of a detection frame provided by the disclosure.

FIG. 4 illustrates a schematic structural diagram of a limit fixed frame provided by the disclosure.

### DESCRIPTION OF REFERENCE NUMERALS

1: bacteria accelerated incubator; 2: support fixed foot; 3: limit fixed frame; 4: limit fixed groove; 5: first fixed pin hole; 6: guide fixed pin; 7: detection frame; 8: processing work case; 9: adjustment control panel; 10: data display device; 11: adjustment control button; 12: limit fixed block; 13: second fixed pin hole; 14: detection positioning bracket; 15: working detection head.

### DETAILED DESCRIPTION OF EMBODIMENTS

The technical solutions in the embodiments of the disclosure will be described clearly and completely below in combination with the drawings in the embodiments of the disclosure. Obviously, the described embodiments are only part of the embodiments of the disclosure, not all of the embodiments. Based on the embodiments of the disclosure, all other embodiments obtained by those skilled in the art without making creative labor belong to the scope of protection of the disclosure.

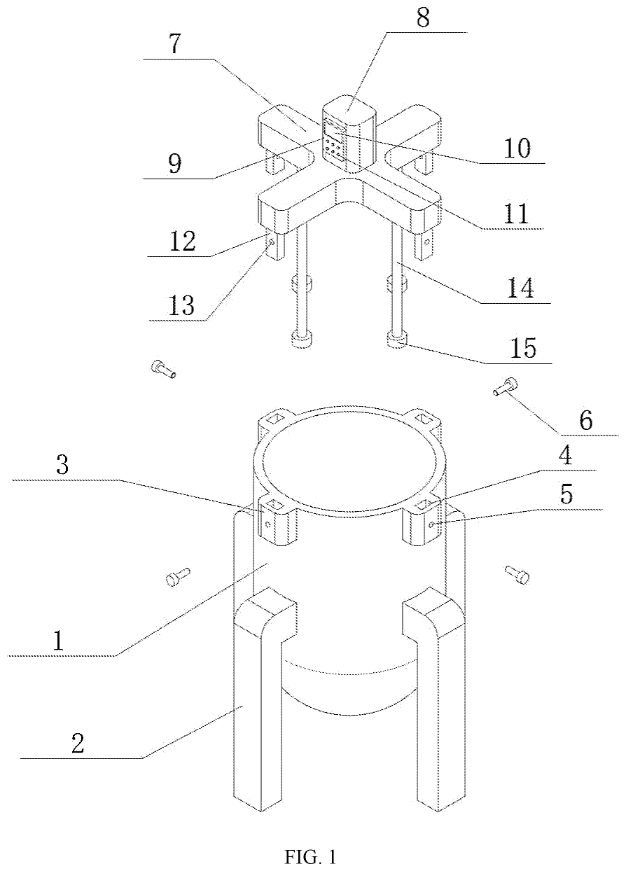

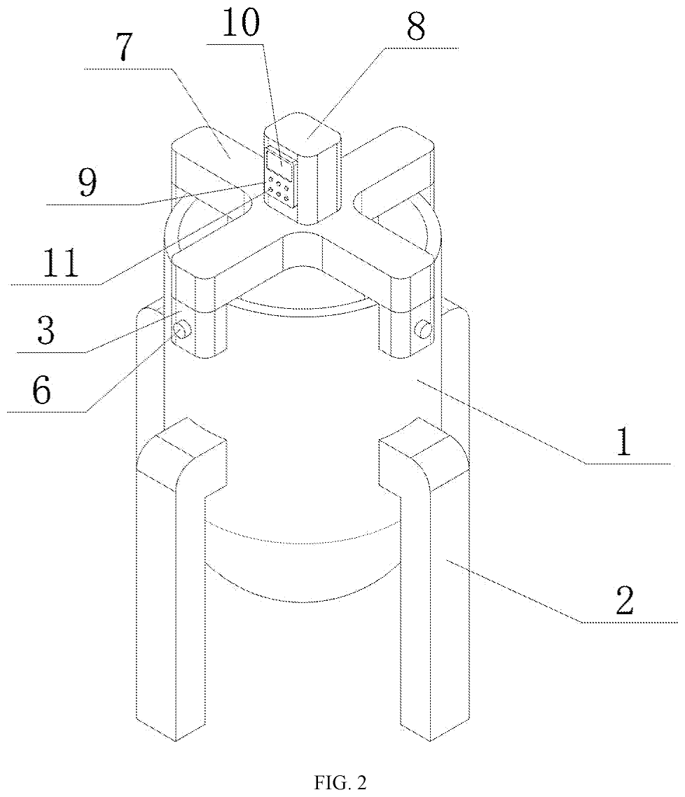

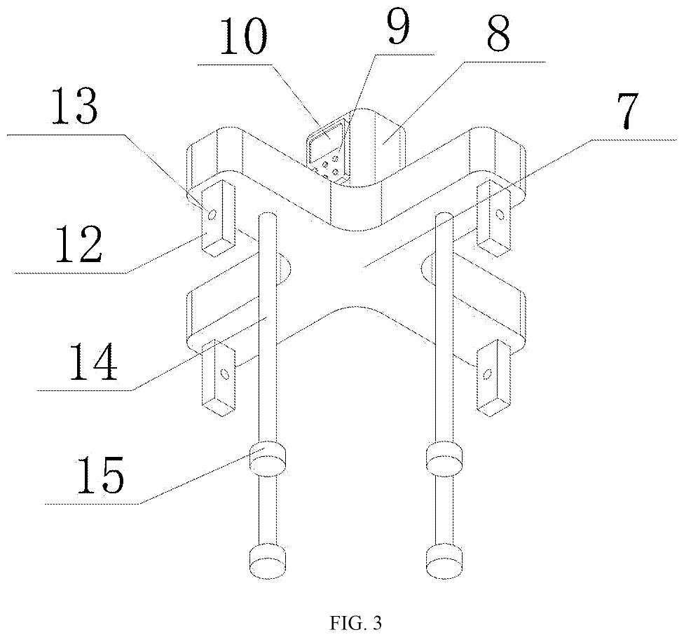

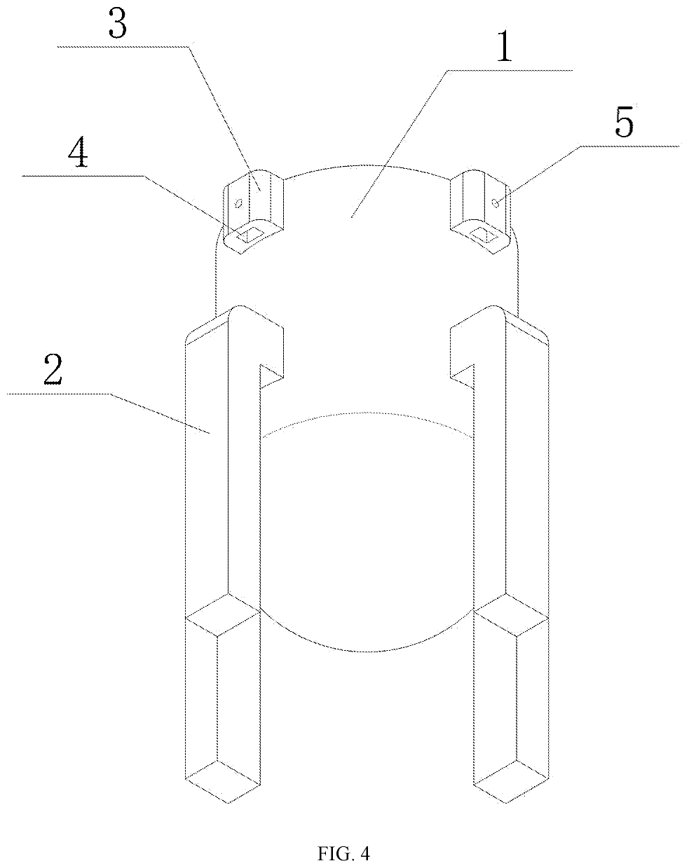

Please refer to FIG. 1 to FIG. 4, in an embodiment of the disclosure, an animal-derived bacteria accelerated culture device with a detection device includes: a bacteria accelerated incubator 1, a lower end of the bacteria accelerated incubator 1 is installed with four support fixed feet 2, an end of the bacteria accelerated incubator 1 facing away from the four support fixed feet 2 is installed with limit fixed frames 3, an upper end of each of the limit fixed frames 3 is provided with a limit fixed groove 4, and an end of each of the limit fixed frames 3 is provided with a first fixed pin hole 5, an upper end of the bacteria accelerated incubator 1 is installed with a detection frame 7, and an end of the detection frame 7 facing away from the bacteria accelerated incubator 1 is installed with a processing work case 8. The working principle of the processing work case 8 is to control working detection heads 15 to detect bacteria inside the bacteria accelerated incubator 1, and detection results are transmitted to the processing work case 8 through detection positioning brackets 14 for analysis and processing, and then data is displayed through a data display device 10, thus the staff can adjust parameters such as corresponding temperature, pH, oxygen, nutrients, etc. of the bacteria accelerated incubator 1 through displayed values, thereby improving work quality and work efficiency.

Specifically, a guide fixed pin 6 is installed inside the first fixed pin hole 5, each the guide fixed pin 6 is matched with the corresponding first fixed pin hole 5, so as to make the detection frame 7 be stably installed on the upper end of the bacteria accelerated incubator 1, thus improving the stability performance.

A front end of the processing work case 8 is installed with an adjustment control panel 9, a front end of the adjustment control panel 9 is installed with a data display device 10, and a lower end of the data display device 10 is installed with adjustment control buttons 11, so as to make an interior of the bacteria accelerated incubator 1 have a good bacteria detection function, thus improving the use performance and work quality.

An end of the detection frame 7 facing away from the processing work case 8 is installed with four limit fixed blocks 12, each the limit fixed block 12 is matched with the corresponding limit fixed groove 4, so as to make the detection frame 7 be stably installed at the upper end of the bacteria accelerated incubator 1, thus improving the stability performance.

An end of each the limit fixed block 12 is provided with a second fixed pin hole 13, and each the second fixed pin hole 13 is matched with the corresponding guide fixed pin 6, so as to make the detection frame 7 be stably installed at the upper end of the bacteria accelerated incubator 1, thus improving the stability performance.

A lower end of the detection frame 7 is installed with four detection positioning brackets 14, each the detection positioning bracket 14 is matched with a depth inside the bacteria accelerated incubator 1, so as to make the interior of the bacteria accelerated incubator 1 have a good bacteria detection function, thus improving the use performance and work quality.

An end of each the detection positioning bracket 14 facing away from the detection frame 7 is installed with a working detection head 15, so as to make the interior of the bacteria accelerated incubator 1 have a good bacteria detection function, thus improving the use performance and work quality.

The working principle of the disclosure is that each the limit fixed block 12 is inserted into the corresponding limit fixed groove 4 at the end of the limit fixed frame 3 until the second fixed pin hole 13 is aligned with the first fixed pin hole 5, and then the corresponding guide fixed pin 6 is inserted through the second fixed pin hole 13 and the first fixed pin hole 5, so as to facilitate to lift the device by fixing adjustment positions and improve the use performance, the working detection heads 15 at the lower end of the detection positioning brackets 14 enter the interior of the bacteria accelerated incubator 1, thus the rapid installation of the detection frame 7 and the bacteria accelerated incubator 1 can be completed, thereby improving the work efficiency. The adjustment control panel 9 at the end of the processing work case 8 can well control the work detection heads 15 to perform real-time detection of the bacteria inside the bacteria accelerated incubator 1, and the data display device 10 can perform data display work well, and the adjustment control buttons 11 can perform adjustment detection work well, thus improving the use performance and work quality.

The above descriptions are only the illustrated embodiments of the disclosure, but the scope of protection of the disclosure is not limited to this. Any person skilled in the art who is familiar with the technical scope of the disclosure and makes equivalent replacement or amendment according to the technical solution and inventive concept of the disclosure within the technical scope of the disclosure shall be covered by the scope of protection of the disclosure.

What is claimed is:

1. An animal-derived bacteria accelerated culture device, comprising:
a bacteria accelerated incubator;
four support fixed feet, installed on a lower end of the bacteria accelerated incubator;
limit fixed frames, installed on an end of the bacteria accelerated incubator facing away from the four support fixed feet; wherein an upper end of each of the limit fixed frames is provided with a limit fixed groove, and an end of each of the limit fixed frames is provided with a first fixed pin hole;
a detection frame, installed on an upper end of the bacteria accelerated incubator; and
a processing work case, installed on an end of the detection frame facing away from the bacteria accelerated incubator.

2. The animal-derived bacteria accelerated culture device according to claim 1, further comprising:
guide fixed pins, installed inside the first fixed pin holes of the limit fixed frames respectively, wherein each of the guide fixed pins is matched with the corresponding first fixed pin hole.

3. The animal-derived bacteria accelerated culture device according to claim 1, further comprising:
an adjustment control panel, installed on a front end of the processing work case;
a data display device, installed on a front end of the adjustment control panel; and
adjustment control buttons, installed on a lower end of the data display device.

4. The animal-derived bacteria accelerated culture device according to claim 1, further comprising:
four limit fixed blocks, installed on an end of the detection frame facing away from the processing work case, wherein the four limit fixed blocks are matched with the limit fixed grooves of the limit fixed frames respectively.

5. The animal-derived bacteria accelerated culture device according to claim 4, wherein an end of each of the limit fixed blocks is provided with a second fixed pin hole, and the second fixed pin holes of the limit fixed blocks are matched with guide fixed pins respectively.

6. The animal-derived bacteria accelerated culture device according to claim 1, further comprising:
four detection positioning brackets, installed on a lower end of the detection frame, and each of the four detection positioning brackets being matched with a depth inside the bacteria accelerated incubator.

7. The animal-derived bacteria accelerated culture device according to claim 6, wherein an end of each of the four detection positioning brackets facing away from the detection frame is installed with a working detection head.

8. An animal-derived bacteria accelerated culture device, comprising:
a bacteria accelerated incubator;
support fixed feet, arranged on an outer peripheral side of the bacteria accelerated incubator, and configured to support the bacteria accelerated incubator;
a detection frame, detachably connected to the bacteria accelerated incubator;
a processing work case, arranged on an end of the detection frame facing away from the bacteria accelerated incubator;
at least one working detection head, arranged on another end opposite to the end of the detection frame and located inside the bacteria accelerated incubator; and
a data display device, arranged above the processing work case;
wherein the at least one working detection head is configured to detect bacteria inside the bacteria accelerated incubator, the processing work case is configured to obtain detection results from the at least one working detection head, and process and analysis the detection results to obtain processed results; and the data display device is configured to display values corresponding to the processed results, so as to adjust parameters of the bacteria accelerated incubator according to displayed values.
